(19) United States
(12) Patent Application Publication
(10) Pub. No.: US 2018/0169327 A1
(43) Pub. Date: Jun. 21, 2018

(54) Fluid Injection Systems and Features

(71) Applicant: Bayer Healthcare LLC, Whippany, NJ (US)

(72) Inventors: Barry Iddon, Jeannette, PA (US); Michael A. Spohn, Fenelton, PA (US); Benjamin James Cullen, Beecroft (AU); Alison Ruth von Moger, Ashfield (AU); Johannes Christoph Behrisch, Munich (DE); Peter John Sprada, Croydon (AU); Richard Sokolov, Earlwood (AU)

(21) Appl. No.: 15/576,303

(22) PCT Filed: May 25, 2016
(86) PCT No.: PCT/US16/34140

§ 371 Date: Nov. 22, 2017

Related U.S. Application Data
(60) Provisional application No. 62/167,003, filed on May 27, 2015.

(51) Int. Cl. A61M 5/145

(52) U.S. Cl. CPC A61M 5/14546; A61M 2205/3553; A61M 2205/3592; A61M 2205/3561; A61M 2205/502

(57) ABSTRACT

A system for delivering fluid to a patient including housing having at least one syringe port and a fluid control device adapted to interface with and actuate at least one syringe engaged therein. The fluid control device includes a processor configured to control a fluid delivery and to wirelessly communicate with one or more portable computers. The portable computer may be programmed or configured to receive one or more fluid delivery parameters from the fluid control device, a user interface, a patient records database, an imaging device, a second computer, a server, a wireless network, or a combination thereof and generate a display containing information pertaining to the fluid delivery parameter based at least partially on the received data. The fluid control device is further configured or programmed to execute one or more control options based at least partially on instructions received from the portable computer.

### CROSS-REFERENCE TO RELATED APPLICATIONS

This application is based on and claims priority to U.S. Provisional Application No. 62/167,003, filed May 27, 2015, which is incorporated herein by reference in its entirety.

### BACKGROUND OF THE DISCLOSURE

### Field of the Disclosure

The present disclosure relates generally to a medical fluid delivery system and apparatus for delivering one or more medical fluids to a patient.

### Description of Related Art

In many medical diagnostic and therapeutic procedures, a medical practitioner, such as a physician, injects a patient with one or more medical fluids. In recent years, a number of injector-actuated syringes and fluid injectors for pressurized injection of medical fluids, such as a contrast solution (often referred to simply as “contrast”), a flushing agent, such as saline, and other medical fluids, have been developed for use in procedures such as angiography, computed tomography (CT), ultrasound, magnetic resonance imaging (MRI), positron emission tomography (PET), and other imaging procedures. In general, these fluid injectors are designed to deliver a preset amount of one or more fluids at a preset pressure and/or flow rate.

In some injection procedures, the medical practitioner places a catheter or a needle connected to tubing, or other fluid delivery connection into a vein or artery of the patient. The catheter or the tubing is connected to either a manual or a powered automatic fluid injection mechanism. Automatic fluid injection mechanisms typically include a connected to a fluid injector having, for example, at least one powered linear piston. The syringe may include a source of contrast and/or a source of flushing fluid. The medical practitioner enters settings into an electronic control system of the fluid injector for a fixed volume of contrast and/or saline, a fixed rate of injection for each, and specific times for injections of each of the one or more fluids.

The injected contrast and/or saline is delivered to a patient's vasculature through the catheter or needle inserted into the patient's body, such as the patient's arm or groin area. A dose of contrast is referred to as a bolus. Once the bolus of contrast is delivered to the desired site, that area is imaged using a conventional imaging technique, such as angiography imaging or scanning, CT, ultrasound, MRI, PET, and/or other imaging procedures. The presence of the contrast becomes clearly visible against the background of the surrounding tissue.

Various front-loading connection interfaces have been developed to facilitate the loading and removal of the syringe to and from the fluid injector. In some embodiments, the syringe having a retention feature is inserted into a syringe port on the fluid injector by aligning the syringe with a corresponding locking feature provided on the fluid injector. It is often necessary for the medical practitioner to manually align the retention feature of the syringe with the corresponding locking feature on the fluid injector before the syringe can be loaded onto the injector. In some cases, there are only one or two possible alignments for loading, such as shown in U.S. Pat. No. 6,336,913. In these syringes, the operator must rotate the syringe to find an alignment that allows the syringe to engage the fluid injector. It is then necessary for the operator to manually rotate the syringe relative to the locking feature to create a strong enough engagement for operation of the injector. In another embodiment disclosed in U.S. Pat. No. 6,652,489, there is no need to rotationally align the syringe or to rotate the syringe for installation or engagement. In other embodiments disclosed in U.S. Pat. Nos. 9,173,995 and 9,199,033, the syringe may rotationally self-align with the locking features of the injector interface upon insertion of the syringe by the user. The disclosures of each of these patents related to front-loading syringe interfaces are incorporated herein by this reference.

Conventional injector design includes a controller, including soft buttons and readouts, located on the face of the injector assembly which requires the user or technician to enter and monitor the injection while remaining within arms-length of the injector assembly. Further, when filling the one or more syringes associated with the fluid injection system, the fluid containers are typically inverted and hung on an adjacent IV stand. However, there is a need for improved injector design that provides benefits and ease of use features to allow a user or technician to freely move throughout the treatment room and readily fill the one or more syringes. While various fluid injection systems and methods are known in the medical field, improved designs and methods for use of the fluid injector which improve the user experience continue to be in demand Particularly, in view of the disadvantages of the existing fluid injection systems that limit user simplicity and experience, there is a need in the art for an improved fluid injection system that provides better user experience and improved workflow.

### SUMMARY OF THE DISCLOSURE

Accordingly, it is an object of the present disclosure to provide a system for delivering fluid to a patient that overcomes some or all of the deficiencies of the prior art. In a non-limiting example of the present disclosure, a system for delivering fluid to a patient, may include an injector housing comprising at least one syringe port adapted to releasably engage at least one syringe; a fluid control device adapted to interface with the injector and actuate at least one syringe engaged in the at least one syringe port, the fluid control device comprising at least one processor programmed or configured to control a fluid delivery and to wirelessly communicate with at least one portable computer such as a laptop computer, tablet computer, smartphone or personal data assistant device, or other handheld computer processor. The at least one portable computer may be programmed or configured to be in wireless and, optionally, wired communication with the at least one processor or injector system and to receive and/or deliver at least one fluid delivery parameter from at least one of the following: the fluid control device, a user interface, an imaging device, a patient records database, a second computer, a server, a hospital network, a wireless network, or any combination thereof and generate a display comprising information pertaining to the at least one fluid delivery parameter based at least partially on the received and/or delivered data. The fluid control device may be configured or programmed to execute at least one control option based at least partially on instructions received and/or delivered from the at least one portable computer.

In another non-limiting example, the housing may be rotatably connected to a support portion and at least one of the housing and the support portion may further include a docking station adapted to releasably engage the at least one portable computer. In some non-limiting examples, the docking station may further comprise a port adapted to interface with the at least one portable computer when the at least one portable computer is engaged in the docking station. The port may be further configured to provide the at least one portable computer with at least one wired connection to at least one of the following: the fluid control device, a user interface, an imaging device, a patient records database, a second computer, a server, a power source, or any combination thereof.

In further non-limiting examples, the docking station may be connected to the housing such that it is rotatable along with the housing relative to the support portion, the display may be generated on at least one of the portable computer and the housing. At least one of the housing, the support portion, and the at least one portable computer may further comprise an orientation sensor configured or programmed to detect an orientation of the at least one portable computer, for example, when it is engaged in the docking station, or, alternatively, while in the disengaged handheld state, and to communicate the orientation to the at least one portable computer, and the at least one portable computer may be programmed or configured to adjust an orientation of the generated display based at least partially on data received from the orientation sensor. The orientation sensor may comprise, but is not limited to, at least one of the following: a gyroscopic sensor, a mechanical sensor, a magnetic sensor, an optical sensor, or any combination thereof, and the at least one portable computer may be programmed or configured to continuously or discontinuously adjust the orientation of the generated display to an upright orientation during a rotation of the housing.

In further non-limiting examples, the at least one portable computer may comprise a circular, elliptical, or oval touch screen and/or a quadrilateral touch screen. The at least one portable computer may be adapted to releasably engage with a docking station attached to the housing, for example, by a magnetic connection or mechanical connection. In certain embodiments, the at least one portable computer may include a rechargeable battery configured to charge when docked with the docking station or when separately connected to a source of electricity.

The at least one control option may comprise, but is not limited to, one or more of the following: initiating a fluid delivery of the one or more fluids, stopping a fluid delivery of the one or more fluids, changing a rate of a fluid delivery of the one or more fluids, recalling and/or executing a pre-programmed fluid delivery protocol of the one or more fluids, delivering a specified volume of the one or more fluids, delivering the one or more fluids in a specified sequence and/or ratio, or any combination thereof.

In additional non-limiting examples, a secondary controller in communication with the fluid control device may be provided, the secondary controller may be connected to or form a part of the housing and be configured or programmed to carry out all or a subset of the at least one control option, including one or more of: initiating a fluid delivery of the one or more fluids and stopping a fluid delivery of the one or more fluids. The secondary controller may be arranged such that when the at least one portable computer is engaged within the docking station, the secondary controller is at least partially physically obscured and/or rendered inoperable.

In further non-limiting examples, the system may further comprise at least one syringe detection unit in communication with one or more of the fluid control devices and the at least one portable computer. The syringe detection unit may be configured or programmed to detect at least one identifying property of the at least one syringe and to communicate the identifying property to one or more of the fluid control device and the at least one portable computer.

In additional non-limiting examples, at least one of the fluid control device and the at least one portable computer may be further configured or programmed to at least one of: prevent a fluid delivery from occurring, provide an alert when a non-compatible, previously used, or non-approved syringe is attached to the at least one syringe port, require a user override when a non-compatible, previously used, or non-approved syringe is attached to the at least one syringe port, and recall at least a portion of a pre-programmed fluid delivery protocol, based at least partially on data received from the syringe detection unit.

The syringe detection unit may comprise, but is not limited to, at least one of the following: a camera, a radio frequency receiver, an optical label reader, a magnetic sensor, an optical sensor, a mechanical sensor, or any combination thereof, wherein at least one of the fluid control device and the at least one portable computer is further configured or programmed to prevent a fluid delivery from occurring unless the at least one identifying property comprises at least one predetermined authorized property or a user authorizes an override to use a non-compatible, previously used, or non-approved syringe.

The at least one identifying property may comprise, but is not limited to, at least one of the following: an indication of the type of syringe, and indication of the brand of the syringe, an indication that the syringe is loaded to a correct volume of fluid, an indication that the syringe has been previously used, an indication whether the syringe has passed its useful shelf-life or expiration date, a lot number or manufacture date or facility, or any combination thereof.

In further non-limiting examples, the system may further include at least one user identifying information detector in communication with and/or integral to at least one of the fluid control device and the at least one portable computer. At least one of the fluid control device and the at least one portable computer may be further configured or programmed to selectively allow an approved, identified user to cause a fluid delivery to occur based at least partially on data received from the at least one user identifying information detector. The user identifying information detector may comprise, but is not limited to, at least one of the following: a biometric scanner, an ID card reader, a microphone, a password protected machine readable-medium, a portable computer identity sensor configured or programmed to determine which of a plurality of portable computers is providing instructions to the fluid control device, or any combination thereof.

In non-limiting embodiments, the system may further comprise at least one proximity sensor in communication with at least one of the fluid control device and the at least one portable computer, and at least one of the fluid control device and the at least one portable computer may be further configured or programmed to prevent a user from initiating a fluid delivery when the at least one portable computer is located a distance further than a safe control distance from the fluid control device based at least partially on information received from the proximity sensor.

In further non-limiting examples, the at least one fluid delivery parameter of the at least one fluid may comprise one or more of the following: a fluid delivery instruction, a fluid delivery flow rate, a fluid delivery duration, a total fluid delivery volume, a remaining volume, a ratio of a first injected fluid to a second injected fluid, a fluid delivery fluid concentration, a fluid delivery fluid identity, a sequence and/or ratio for delivering at least two fluids, a pre-programmed fluid delivery protocol, or any combination thereof.

Additionally, it is an object of the present disclosure to provide an apparatus for delivering fluid to a patient that overcomes some or all of the deficiencies of the prior art. According to another non-limiting example, provided is an injector assembly, comprising a housing comprising at least one syringe port adapted to releasably engage at least one syringe; a fluid control device adapted to interface with and actuate at least one syringe engaged in the at least one syringe port, the fluid control device comprising at least one processor programmed or configured to control a fluid delivery and to communicate with at least one portable computer; and a support portion comprising a retractable pole configured to reversibly move from a retracted position to a deployed position to extend above the injector assembly, the retractable pole comprising at least one hanging feature for hanging at least one multi-dose fluid container. In other non-limiting examples, the support portion may further comprise a catch adapted to reversibly engage with the retractable pole and releasably lock the retractable pole into at least one locked position.

The at least one hanging feature may comprise, but is not limited to, at least one of the following: a pivoting hook which is pivotable from a retracted position, wherein at least a portion of the pivoting hook is substantially contained within a support member of the retractable pole, to a deployed position wherein the at least a portion of the pivotable hook extends away from the support member, and a rotatable hook which is rotatable about the longitudinal axis of a support member of the retractable pole, or any combination thereof. In the deployed position, the pivotable hook and/or rotatable hook is capable of holding the at least one multi-dose fluid container, hanging above at least one syringe in the syringe port of the injector assembly.

In non-limiting examples, the retractable pole may further comprise a handle portion which extends above at least one of the housing and the support portion when the retractable pole is in a retracted position. Alternatively, the handle portion may be flush with the surface of the housing or support portion, when in the retracted position, but may further include a gripping surface for gripping the retractable pole to move the pole into the deployed position. In the retracted position, the retractable pole reduces the space requirements for the injector system and eliminates the need for a separate hanging apparatus, for example an IV pole.

The at least one multi-dose fluid container may be selected from but is not limited to a saline bag, a multi-dose pharmaceutical liquid container, a container comprising multiple doses of a contrast or imaging agent, or any combination thereof.

In various examples, the present disclosure may be characterized by one or more of the following clauses:

Clause 1. A system for delivering fluid to a patient, the system comprising: an injector housing comprising at least one syringe port adapted to releasably engage at least one syringe; a fluid control device adapted to interface with and actuate at the least one syringe engaged in the at least one syringe port, the fluid control device comprising at least one processor programmed or configured to control a fluid delivery of at least one fluid to at least one patient and to communicate with at least one portable computer; wherein the at least one portable computer is programmed or configured to receive or deliver at least one fluid delivery parameter from or to at least one of the following: the fluid control device, a user interface, an imaging device, a patient records database, a second computer, a server, a hospital network, a wireless network, or any combination thereof and generate a display comprising information pertaining to the at least one fluid delivery parameter based at least partially on the received and/or delivered data; and wherein the fluid control device is configured or programmed to execute at least one control option based at least partially on instructions received and/or delivered from the at least one portable computer.

Clause 2. The system according to clause 1, wherein the at least one portable computer comprises at least one of a laptop computer, a tablet computer, a smartphone, a personal data assistant device, a handheld computer, or any combination thereof.

Clause 3. The system according to clauses 1 or 2, wherein the at least one portable computer is further configured to be in wireless and, optionally, wired, communication with the fluid control device.

Clause 4. The system according to any of clauses 1-3, wherein the housing is rotatably connected to a support portion and wherein at least one of the housing and the support portion further comprises a docking station adapted to releasably engage the at least one portable computer.

Clause 5. The system according to clause 4, wherein the docking station further comprises a port adapted to interface with the at least one portable computer when the at least one portable computer is engaged in the docking station and to provide the at least one portable computer with at least one wired connection to at least one of the following: the fluid control device, a user interface, an imaging device, a patient records database, a second computer, a server, a power source, or any combination thereof.

Clause 6. The system according to clauses 4 or 5, wherein: the docking station is connected to the housing such that it is rotatable along with the housing relative to the support portion; the display is generated on at least one of the portable computer and the housing; at least one of the housing, the support portion, and the at least one portable computer further comprises an orientation sensor configured or programmed to detect an orientation of the at least one portable computer at least while the at least one portable computer is engaged in the docking station, or, alternatively, while the at least one portable computer is in a disengaged, handheld state, and to communicate the orientation to the at least one portable computer, and wherein the at least one portable computer is programmed or configured to adjust an orientation of the generated display based at least partially on data received from the orientation sensor.

Clause 7. The system according to clause 6, wherein the orientation sensor comprises at least one of the following: a gyroscopic sensor, a mechanical sensor, a magnetic sensor, an optical sensor, or any combination thereof, and wherein the at least one portable computer is programmed or configured to continuously or discontinuously adjust the orientation of the generated display to an upright orientation during a rotation of the housing.

Clause 8. The system according to any of clauses 1-7, wherein the at least one portable computer comprises at least one of a circular touch screen, an oval touch screen, an elliptical touch screen, and a quadrilateral touch screen.

Clause 9. The system according to any of clauses 1-8, wherein the at least one portable computer is adapted to releasably engage with a docking station attached to the housing via at least one of a mechanical connection and a magnetic connection.

Clause 10. The system according to any of clauses 1-9, wherein the at least one portable computer further comprises a rechargeable battery configured to charge when the at least one portable computer is engaged with the docking station.

Clause 11. The system according to any of claims 1-10, wherein: the at least one control option comprises at least one of the following: initiating a delivery of the at least one fluid, stopping a delivery of the at least one fluid, changing a rate of a delivery of the at least one fluid, recalling and/or executing a pre-programmed fluid delivery protocol of the at least one fluid, delivering a specified volume of the at least one fluid, delivering the at least one fluid in a specified sequence and/or ratio, or any combination thereof.

Clause 12. The system according to any of clauses 1-11, further comprising: a secondary controller in communication with the fluid control device; wherein the secondary controller is connected to or forms a part of the housing; and wherein, the secondary controller is configured or programmed to carry out at least a subset of the at least one control option, including at least one of initiating a delivery of the least one fluid and stopping a delivery of the at least one fluid.

Clause 13. The system according to clause 12, wherein the secondary controller is arranged such that when the at least one portable computer is engaged within the docking station, the secondary controller is at least partially physically obscured and/or rendered inoperable.

Clause 14. The system according to any of clauses 1-13, further comprising at least one syringe detection unit in communication with at least one of the fluid control device and the at least one portable computer, wherein the syringe detection unit is configured or programmed to detect at least one identifying property of the at least one syringe and to communicate the at least one identifying property to at least one of the fluid control device and the at least one portable computer.

Clause 15. The system according to clause 14, wherein at least one of the fluid control device and the at least one portable computer is further configured or programmed to at least one of: prevent a fluid delivery from occurring, provide an alert when a non-compatible, previously used, or non-approved syringe is attached to the at least one syringe port, require a user override when a non-compatible, previously used, or non-approved syringe is attached to the at least one syringe port, and recall at least a portion of a pre-programmed fluid delivery protocol, based at least partially on data received from the syringe detection unit, or any combination thereof.

Clause 16. The system according to any of clauses 14-15, wherein the syringe detection unit comprises at least one of the following: a camera, a radio frequency receiver, an optical label reader, a magnetic sensor, an optical sensor, a mechanical sensor, or any combination thereof, wherein at least one of the fluid control device and the at least one portable computer is further configured or programmed to prevent a fluid delivery from occurring unless the at least one identifying property comprises at least one predetermined authorized property or an override command is received from an authorized user.

Clause 17. The system according to any of clauses 14-16, wherein the at least one identifying property comprises at least one of the following: an indication of the type of syringe, and indication of the brand of the syringe, an indication that the syringe is loaded to a correct volume of fluid, an indication that the syringe has been previously used, an indication whether the syringe has passed its useful shelf-life, an expiration date, a lot number, a manufacture date, a facility of manufacture, or any combination thereof.

Clause 18. The system according to any of clauses 1-17, further comprising at least one user identifying information detector in communication with and/or integral to at least one of the fluid control device and the at least one portable computer, wherein at least one of the fluid control device and the at least one portable computer is further configured or programmed to selectively allow an approved, identified user to cause a fluid delivery to occur based at least partially on data received from the at least one user identifying information detector.

Clause 19. The system according to clauses 18, wherein the user identifying information detector comprises at least one of the following: a biometric scanner, an ID card reader, a microphone, a password protected machine readable-medium, a portable computer identity sensor configured or programmed to determine which of a plurality of portable computers is providing instructions to the fluid control device, or any combination thereof.

Clause 20. The system according to any of clauses 1-19, further comprising a proximity sensor in communication with at least one of the fluid control device and the at least one portable computer, wherein at least one of the fluid control device and the at least one portable computer is further configured or programmed to generate at least one warning and/or to prevent a user from initiating a fluid delivery when the at least one portable computer is located a distance further than a safe control distance from the fluid control device based at least partially on information received from the proximity sensor.

Clause 21. The system according to any of clauses 1-20, wherein the at least one fluid delivery parameter comprises one or more of the following: a fluid delivery instruction, a fluid delivery flow rate, a fluid delivery duration, a total fluid delivery volume, a remaining volume, a ratio of a first injected fluid to a second injected fluid, a fluid delivery fluid concentration, a fluid delivery fluid identity, a sequence and/or ratio for delivering at least two fluids, a pre-programmed fluid delivery protocol, or any combination thereof.

Clause 22. An injector assembly, comprising a housing comprising at least one syringe port adapted to releasably engage at least one syringe; a fluid control device adapted to interface with and actuate at least one syringe engaged in the at least one syringe port, the fluid control device comprising at least one processor programmed or configured to control a fluid delivery and to communicate with at least one portable computer; and a support portion comprising a retractable pole configured to reversibly move from a retracted position to a deployed position to extend above the injector assembly, the retractable pole comprising at least one hanging feature for hanging at least one multi-dose fluid container.

Clause 23. The injector assembly according to clause 22, wherein: the support portion further comprises a catch adapted to reversibly engage with the retractable pole and releasably lock the retractable pole into at least one locked position; wherein the at least one hanging feature comprises at least one pivotable hook which is pivotable from a retracted position, wherein at least a portion of the pivotable hook is substantially contained within a support member of the retractable pole, to a deployed position wherein the at least a portion of the pivotable hook extends away from the support member; wherein the at least one multi-dose fluid container is selected from a saline bag, a multi-dose pharmaceutical liquid container, a container comprising multiple doses of a contrast or imaging agent, or any combination thereof; and wherein the at least one hanging feature is capable of supporting the at least one multi-dose fluid container when the at least one hanging feature is in the deployed position.

Clause 24. The injector assembly according to clause 22 or 23, further comprising: a rotatable tray member which is rotatable about the longitudinal axis of a first support member of the retractable pole, from a retracted position, wherein at least a portion of the rotatable tray member is stored substantially between the first support member and the second support member, to a deployed position wherein at least a portion of the rotatable tray member extends away from the first support member.

While several examples of fluid injection and delivery systems are shown in the accompanying figures and described herein in detail, other examples will be apparent to, and readily made by, those skilled in the art without departing from the scope and spirit of the disclosure. For example, it is to be understood that this disclosure contemplates that, to the extent possible, one or more features of any example can be combined with one or more features of any other example. Accordingly, the foregoing description is intended to be illustrative rather than restrictive.

### BRIEF DESCRIPTION OF THE DRAWINGS

FIG. 1. illustrates a fluid injection system including a detachable portable computer according to a non-limiting example of the present disclosure.

FIG. 2. illustrates a fluid injection system as a mobile work station including a detachable portable computer according to one non-limiting example of the present disclosure.

FIG. 3. illustrates a fluid injection system including a detachable portable computer in a detached configuration according to one non-limiting example of the present disclosure.

FIG. 4. illustrates a detachable touch screen for a fluid injection system according to one non-limiting example of the present disclosure.

FIGS. 5A-5D illustrate a retractable pole for a fluid injection system in a retracted position, in a partially extended position, and in a fully extended position according to a non-limiting example of the present disclosure.

FIG. 6. illustrates a retractable pole for a fluid injection system in the fully extended operational position according to various non-limiting examples of the present disclosure.

FIG. 7. illustrates a fluid delivery system diagram in accordance with a non-limiting example of the present disclosure.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to an injector, the term “proximal” refers to a portion of an injector furthest from a syringe port of an injector. The term “distal” refers to a portion of an injector closest to a syringe port of an injector. It is to be understood, however, that the disclosure may assume alternative variations and step sequences, except where expressly specified to the contrary. It is also to be understood that the specific devices and processes illustrated in the attached drawings, and described in the following specification, are simply illustrative examples of the disclosure. Hence, specific dimensions and other physical characteristics related to the examples (i.e., aspects, variants, variations) disclosed herein are not to be considered as limiting.

As used herein, the terms “communication” and “communicate” refer to the receipt or transfer of one or more signals, messages, commands, or other type of data. For one unit or device to be in communication with one or more other unit or device means that the one unit or device is able to receive data from and/or transmit data to the one or more other unit or device. A communication may use a direct or indirect connection, and may be wired and/or wireless in nature. Additionally, two or more units or devices may be in communication with each other even though the data transmitted may be modified, processed, routed, etc., between the first and second unit or device. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. In non-limiting examples, a communication may occur through one or more wired or wireless connections, such as, through one or more wires, through direct wireless protocols such as Bluetooth, Near Field Communication (NFC), or other radio frequency protocols, and/or through indirect wireless communication such as through a local Wi-Fi network or secure Internet connection. Wireless communication may include, but is not limited to, any communication that does not require direct wired contact between the two communicating units or devices such as via a Wi-Fi network, communication via Bluetooth, NFC, or other conventional wireless system, or other non-wired electromagnetic communication systems. It will be appreciated that numerous other arrangements are possible.

In another non-limiting example, a fluid injection system is provided, having an injector assembly with at least one syringe port, a controller, and a wired or wirelessly connected portable computer, such as a reversibly detachable portable computer, which may have a touch screen or other user input device, and/or a display configured for inputting one or more injection parameters and displaying one or more injection features. Suitable detachable portable computers may include, but are not limited to, a laptop computer, a tablet computer, a smartphone or a personal data assistant device, or other handheld computer processor. The portable computer may be in communication with the controller by a wired or wireless communication mechanism.

In additional examples, a fluid injection system may have an injector assembly with at least one syringe port and, optionally, a detachable portable computer, wherein the injector assembly further includes a retractable pole configured to extend above the injector assembly and having at least one hanging feature for hanging at least one multi-dose fluid container. The multi-dose fluid container can be a saline bag or container, a multi-dose pharmaceutical liquid container, a container containing multiple doses of a contrast or imaging agent, and various combinations thereof.

Various examples of the present disclosure are directed to fluid injection systems for injecting one or more fluids into a patient during an injection procedure. The design and features of the fluid injection system provide ease of use and improved user experience, thereby allowing for closer monitoring of an injection procedure and the ability to move throughout an injection suite during the injection procedure while being able to control one or more parameters or features of the ongoing injection procedure. Further features allow for hanging of one or more multi-dose fluid containers on the fluid injection system for ready access when filling and/or refilling the one or more syringes associated with the fluid injection system. In particular examples, the fluid injection systems of the present disclosure may be used for injection of one or more imaging agents in an imaging procedure, such as, for example CT scan, MRI, and other radiological imaging procedures. The various examples of the fluid injection systems may comprise an injector assembly comprising at least one syringe port for interfacing with a syringe and be configured to inject one or more medical fluids during an imaging procedure.

In specific examples, the fluid injector system may be a front-loading fluid injector system similar to the various examples of the injectors disclosed in U.S. Pat. Nos. 5,383,858, 7,553,294, 7,666,169, 9,173,995 9,199,033 and in International Patent Application No. PCT/US2012/0374891, and in U.S. Patent Application Publication No. 2014/0027009, the disclosures of which are incorporated by reference in their entirety. Other examples may include new fluid injector systems designed to include various examples of the interface described herein.

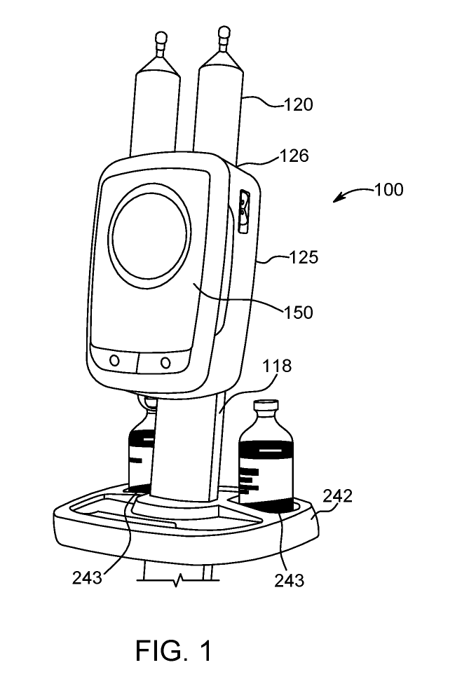

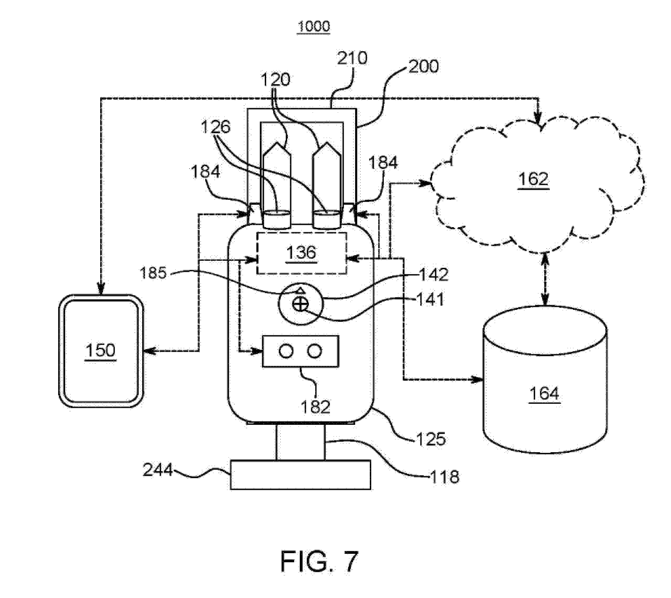

illustrates a fluid injection system including a detachable portable computer 150 comprising a touch screen, according to a non-limiting example of the present disclosure. With reference to FIG. 1, a fluid delivery device or injector 100 (hereinafter referred to as “injector 100”), such as an automated or powered fluid injector, is adapted to interface with and actuate at least one syringe 120, which may be independently filled with a medical fluid, such as contrast media having a desired concentration or identity, saline solution, or other desired medical fluids. The injector 100 may be used during a medical procedure, such as an imaging procedure, to inject the medical fluid into the body of a patient by driving a plunger (not shown) of the at least one syringe 120 with at least one piston (not shown) operated by a fluid control device (not shown; 136 in FIG. 7), which may be at least partially internal to the injector 100. In non-limiting examples, the injector 100 may be a multi-syringe injector, wherein a plurality of syringes 120 may be oriented side-by-side or in another arrangement and include plungers separately actuated by respective pistons associated with the injector 100, and controlled by the fluid control device. In one non-limiting example, two syringes may be arranged in a side-by-side fashion and filled with two different medical fluids, such as a contrast agent and a saline solution, and the injector 100 may be configured to deliver fluid to a patient from one or both of the syringes 120 either sequentially or simultaneously. It will be appreciated that various other arrangements are possible.

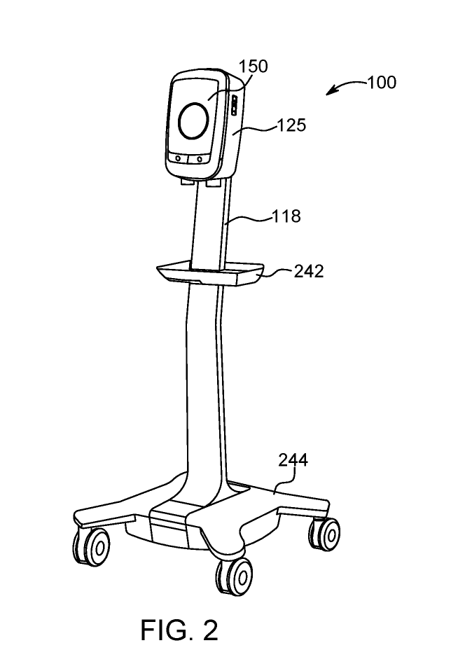

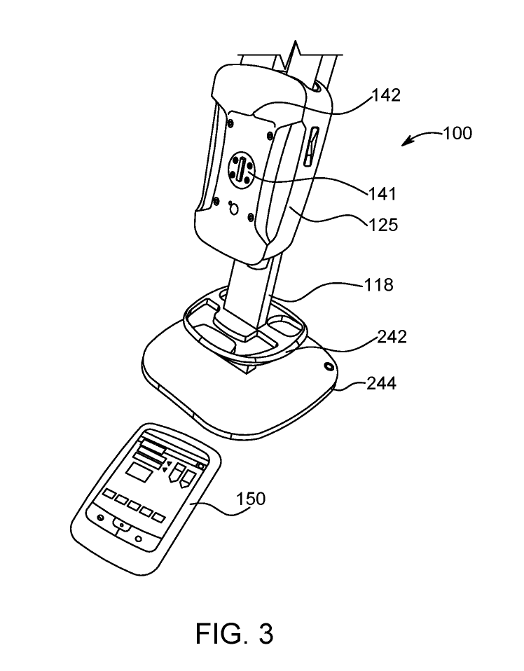

The injector 100 may have a housing 125 formed from a suitable structural material, such as plastic, a composite material, and/or metal. The housing 125 may be of various shapes and sizes depending on the desired application. For example, the injector 100 may be a freestanding structure having a support portion 118 connected to a base 244 with one or more rollers or wheels such that the injector 100 is movable over the floor (see FIG. 2). In other examples, the injector 100 may have smaller design for placement on a suitable table or support frame (see FIG. 3). The injector 100 may include at least one syringe port 126 for releasably connecting the at least one syringe 120 to respective piston elements. In various examples, the at least one syringe 120 includes at least one syringe retaining member (not shown) configured for retaining the syringe 120 within the syringe port 126 of the injector 100. In non-limiting examples, the at least one syringe retaining member is configured to operatively engage a locking mechanism provided on or in the syringe port 126 of the injector 100 to facilitate self-oriented loading and/or removal of the syringe 120 to and from the injector 100. The syringe retaining member and the locking mechanism together define a connection interface for connecting the syringe 120 to the injector 100.

In non-limiting examples, at least one fluid path set (not shown) may be fluidly connected with the distal end of the at least one syringe 120 for delivering medical fluid from the at least one syringe 120 to a catheter, needle, or other fluid delivery connection (not shown) inserted into a patient at a vascular access site. Fluid flow from the at least one syringe 120 may be regulated by a fluid control module operated by a controller, such as a detachable touch screen controller 150 or any suitable device. The fluid control module may operate various, pistons, valves, and/or flow regulating devices to regulate the delivery of the medical fluid, such as saline solution and contrast, to the patient based on one or more user selected injection parameters, such as injection flow rate, duration, total injection volume, and/or ratio of contrast media and saline.

In further non-limiting examples, the injector 100 may be a part of a larger fluid delivery system, wherein a fluid delivery device 136 of the injector 100 may be in communication with one or more of a portable computer 150, a network 162, a patient records database 164, a plurality of syringe identification sensors 184, and a secondary controller 182 (See FIG. 7). In non-limiting examples, the fluid control device 136 and/or portable computer 150 may be further in communication with a medical scanner or imager, an injection protocol database, or other system associated with the injection procedure.

Having generally described the structure and function of the fluid injector system 100, the various features associated with the present disclosure will now be described in reference to the Figures. FIG. 1 illustrates an injector 100 according to a non-limiting example of the present disclosure. The injector 100 may include an internal fluid control device (not shown in FIG. 1, see 136 in FIG. 7) which may be controlled by the portable computer 150.

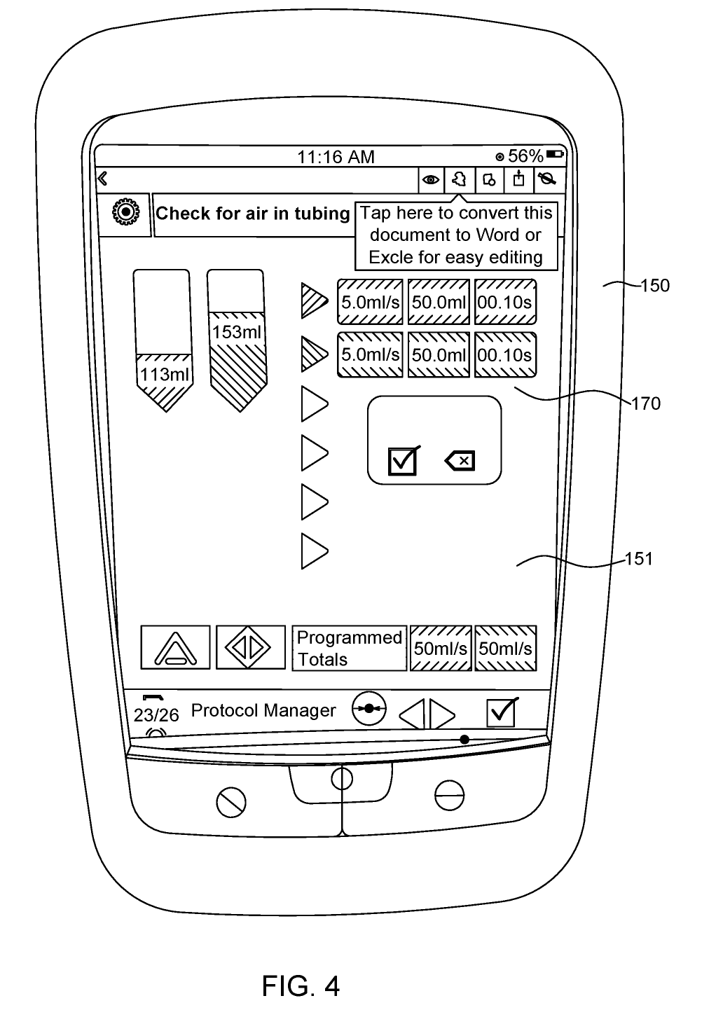

The portable computer may include one or more processors, memory, network interfaces, and/or the like and may be configured to generate a display comprising a graphical user interface (“GUI”), which may allow a user to view and/or interact with various injection parameters through graphical icons and visual indicators produced on the display (see, e.g. FIG. 4). In non-limiting examples, the portable computer 150 may be formed as a detachable touch screen controller. The portable computer 150 may be used to monitor one or more injection parameters, including, for example, patient specific information (age, weight, sex, organ to be imaged, dosage of imaging agent, etc.), which may be inputted by the user or recalled/downloaded from a database 164, a network 162, a memory, or another computer in communication with the system by a wired or wireless communication process. The portable computer 150 may be further configured to control various injection parameters which may be inputted by a user and/or calculated by one or more algorithmic calculations performed by the portable computer 150, the fluid control device (136 in FIG. 7), and/or another computer or processor in communication with the fluid control device and/or the portable computer based on data downloaded from a database and/or inputted by a user.

Various user selected injection parameters 170 (see FIG. 4) and injection instructions, such as injection flow rate, injection start time, duration, total injection volume of each of the one or more fluids, remaining volume to be injected, ratio of injected fluid, volume of one or more fluid remaining in a multi-dose medical fluid container after the injection procedure, and various other parameters associated with the contrast media and saline injection fluids may be displayed on the touch screen of the portable computer 150 and may be manipulated, viewed, or recorded as required by the user by at least one input or output mechanism, for example, changing parameters by utilizing the touch screen and/or one or more additional computers in communication with the portable computer 150. The portable computer 150 may also display alerts or other information determined by the fluid control device or the injection system to notify a user of an event, such as a low fluid level of a syringe connected to an injector port, the size or type of syringe detected to be in the injector port, the fluid volume of one or more syringes, the contents of one or more syringes, air detection in one or more syringes, an indication that the syringe has been previously used, an indication whether the syringe has passed its useful shelf-life expiration date, a lot number or manufacture date or facility, etc.

In the non-limiting example shown, the portable computer 150 is detachable from the injector housing 125 and may be utilized to operate the fluid injection system remotely from other positions in the room or, alternatively, while attached to the injector housing 125. The portable computer 150 may be in communication with the fluid injection system through one or more wired or wireless communication connections, such as through one or more wires, through direct wireless protocols such as Bluetooth, Near Field Communication (NFC) or other radio frequency protocols, and/or through indirect wireless communication, such as through a local Wi-Fi network or secure Internet connection. However, it will be appreciated that various wired and wireless communication mechanisms may be used in accordance with the present disclosure.

According to various examples, wireless communication between the portable computer 150 and the fluid control device 136 affords the user freedom to move around the injection suite with the portable computer 150 while continually monitoring the patient and injection procedure. Thus, the user may move to address various aspects of an injection procedure that are out of arms-reach from the injector housing 125 while still maintaining control over the injection procedure. The portable computer 150 may dock with the injector assembly (compare FIGS. 1 and 2, showing the portable computer in the docked position, with FIG. 3, showing the portable computer 150 in the undocked and detached position). According to various examples, the portable computer 150 may comprise an internal, rechargeable battery, and may be configured to charge or replenish its electrical charge while docked with the injector 100, via one or more ports 141 which may provide electrical connections between the portable computer 150 and injector 100. Additionally or alternatively, the portable computer 150 may be charged by a standalone charging station or computer, or by connection to an electrical outlet.

With continued reference to FIG. 1, at least one feature such as a medical fluid tray 242 may be provided to facilitate the efficient handling and/or storage of medical fluids or various disposable items for the injection procedure, such as disposable tubing sets, syringes, etc. In the non-limiting example shown, the medical fluid tray is connected to a support portion 118 of the injector 100 and is adapted to house a plurality of medical fluid bottles 243.

Referring to FIG. 2, a non-limiting example of the injector 100 is illustrated as a standing workstation with the detachable portable computer 150 in the attached position, wherein the support portion 118 is mounted to a base 244 comprising wheels to allow free movement of the injector 100 throughout the injection suite or between rooms. The combination of the detachable portable computer 150 with a movable standing workstation provides additional freedom of movement for a user to move the fluid injection system near the patient, prepare the injection procedure, and then move around the injection suite or behind shielding to reduce radiation exposure while monitoring the injection with the portable computer 150.

FIG. 3 shows a non-limiting example of the fluid injection system 100 with the portable computer 150 having a touch screen in the detached configuration. In the non-limiting example shown, wireless communication between portable computer 150 and the fluid control device 136 (internal to the housing 125) allows for continuous communication between the user and the fluid control device and facilitates the rapid monitoring and changing of input parameters for an injection protocol. Operation of the injection device may include use of any combination of the touch screen, soft-touch keys, and hard-touch keys located on the portable computer 150, the injection device, or a wired local operation station (not shown). In certain embodiments, one or more images from the scanner may be displayed on the portable computer 150 for view by the user. For example, test images to help determine the correct orientation or placement of the patient within the scanner, or to locate or confirm that the bolus of contrast has reached the site of interest, may be taken to enhance the imaging process. Further, in certain embodiments, images may be displayed on the portable computer to allow the user to confirm that the imaging procedure has been successful, that image clarity is sufficient, and/or to allow quick analysis of the image.

FIG. 4 shows a GUI 151 of a portable computer 150 in accordance with a non-limiting example of the present disclosure, showing various injection parameters 170 for an injection procedure, displayed on a touch screen. As described herein, the various parameters 170 may be monitored, changed, or inputted by the user before, during, or after an injection procedure, for example by touching the appropriate field on the touch screen and entering the appropriate data using an electronic keypad that appears on the touch screen. In certain examples, some or all of the parameters may be saved and uploaded to a patient records database either wirelessly or by wired connection from the portable computer or another computer, for example a hospital information system or network. In non-limiting configurations, various parameters may be saved and uploaded automatically and/or in response to a user command.

According to other non-limiting examples, the fluid injection systems of the present disclosure may comprise an extendible/retractable pole 200 configured to extend above the injector housing 125 and having at least one hanging feature such as, for example, one or more pivotable hooks 250 for hanging at least one container such as a multi-dose fluid container. According to various examples, the fluid injection systems of the present disclosure may be used for multiple sequential injection procedures, wherein the system includes a multi-patient portion and a single-patient portion. In these examples, the at least one syringe 120 may be refilled with the appropriate medical fluid between fluid injection procedures from at least one multi-dose fluid container, such as a saline bag or container, a contrast bottle, or other appropriate medical container.

For ease of use and convenience, the at least one multi-dose fluid container may be suspended in an inverted position above the housing 125 from a hanging feature (e.g., 250, 260) such as a hook, tray, or other protrusion, while filling or refilling the at least one syringe 120 or during the injection procedure where the fluid container is not in fluid communication with the at least one syringe 120. Thus, the multi-dose fluid containers may be readily available after an injection procedure to refill the at least one syringe 120 and prepare the fluid injection system for a subsequent injection procedure.

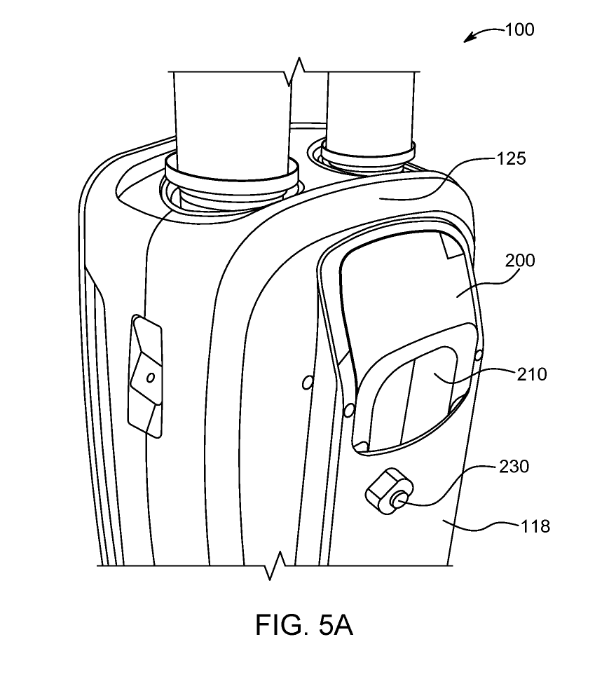

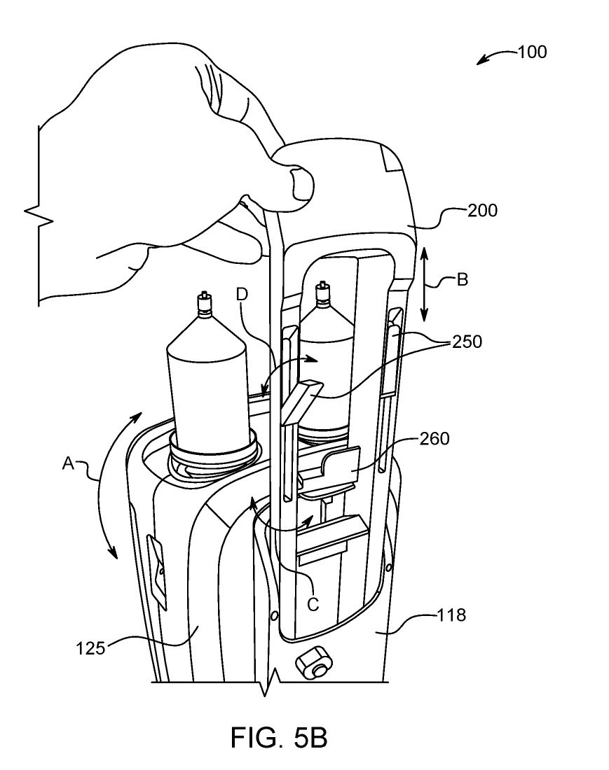

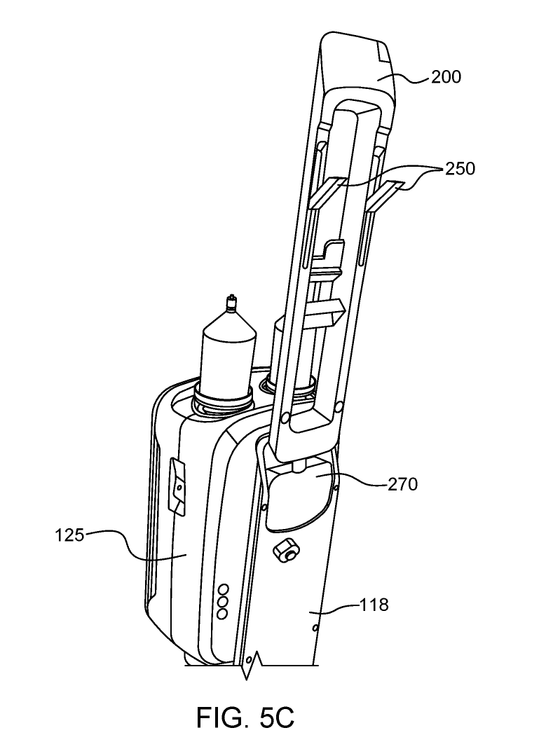

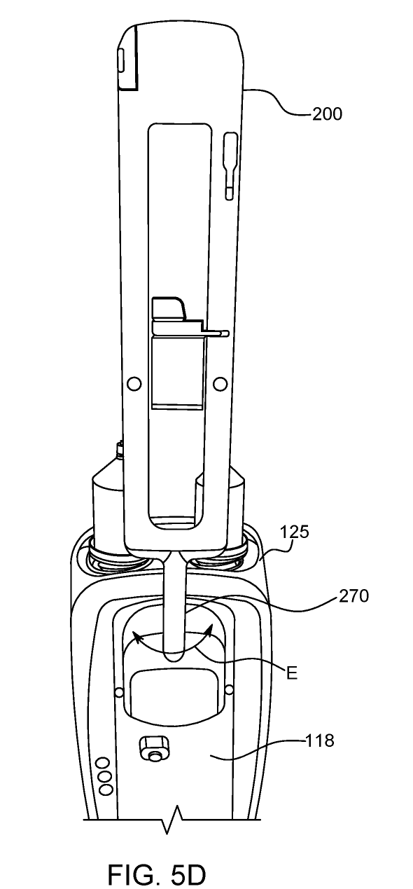

FIGS. 5A-5D show an injector 100 having a retractable pole 200 according to a non-limiting example of the fluid injection system of the present disclosure. Referring to FIG. 5A, the retractable pole 200 is shown in a retracted position for storage and to minimize the footprint of fluid injection system 100 as well as eliminate the requirement for a separate rollable IV pole to hang the one or more medical fluid containers. The retractable pole includes a handle 210 or other gripping surface configured for gripping by a user and to facilitate the extension of the pole 200 upwards above the support portion 118 (see Arrow B in FIG. 5B). In certain examples, the extendible pole may be held or locked in a specific position that may be disengaged by pressing button 230 before or concurrently with moving handle 210.

FIG. 5B shows an injector 100 with the retractable pole 200 in a partially extended position. In the non-limiting example shown, the retractable pole 200 is provided with a plurality of pivotable hooks 250 with at least one hook 250 being pivotable from a retracted position, wherein the pivotable hook is substantially contained within a support member of the retractable pole 200, to a deployed position wherein the at least a portion of the pivotable hook extends away from the associated support member (see Arrow C in FIG. 5B). Additionally, in the non-limiting example shown, the retractable pole 200 further includes a rotatable tray member 260 which is rotatable about the longitudinal axis of a support member of the retractable pole from a position where it is stored substantially between the support members to a deployed position to provide increased accessibility for positioning a multi-dose fluid container thereon (see Arrow D in FIG. 5B).

In certain non-limiting examples, it is desirable to temporarily rotate and/or invert the injector housing 125 including syringe ports 126 between a substantially vertical position (i.e., with the syringe port(s) pointing upwards), which may facilitate, for example, the loading of a syringe 120 into a syringe port 126 or the filling of a syringe with medical fluid, and an inverted position, which may facilitate, for example, the removal of air bubbles in a medical fluid contained within a syringe 120, or the conducting of an injection procedure. Accordingly, in non-limiting examples of the present disclosure, the housing 125 may be connected to the support portion 118 in a rotatable fashion (see Arrow A in FIG. 5B) such that the housing is rotatable relative to the support portion 118 and retractable pole 200.

The herein-described rotation of the housing 125 relative to the support portion 118 causes the portable computer 150, when engaged in a docking station 142 on the housing 125, to rotate along with the housing 125. Accordingly, in certain non-limiting examples, at least one of the housing 125, the support portion 118, the docking station 142, and the at least one portable computer 150 further comprises an orientation sensor (185 in FIG. 7) configured or programmed to detect an orientation of the at least one portable computer when it is engaged in the docking station, and, optionally, while in the disengaged state, and to communicate the orientation to the at least one portable computer. Various orientation sensors may be used in accordance with the present disclosure, such as, for example, a gyroscopic sensor, a mechanical sensor, a magnetic sensor, an optical sensor, or any combination thereof. The orientation may be detected using various parameters including but not limited to a detection of a change in the direction of gravity, the rotation of an image from a known point as detected by an optical sensor or camera, and/or the degree of rotation of the housing from a known point, as detected, for example, by a mechanical or magnetic sensor.

The portable computer 150 may be configured or programmed to adjust the orientation of its display including the various touch pad features, to an upright rotation throughout the rotation of the housing in a continuous or discontinuous fashion based on data from the orientation sensor 185, even when the display is rotated at non-90° angles. The user experience may be further improved to provide near-seamless image viewing/control by providing the portable computer with a physical or virtual touch screen. For example, the touch screen of the portable computer may be shaped in a circular fashion, or a virtual circular interface may be displayed on a quadrilateral or other shaped touch screens and the portable computer may be configured or programmed to continuously or discontinuously adjust the image within the virtual circular interface to an upright orientation throughout a rotation of the housing based at least partially on information received from the orientation sensor 185.

FIG. 5C shows the retractable pole 200 in an extended position with hanging features 250 in a deployed state. In non-limiting examples, the pole 200 may include two rotation positions, a storage position, and a use position (shown in FIG. 5D). According to this example, the retractable pole 200 may rotate around a point 270 (see Arrow E in FIG. 5D) to move the retractable pole 200 from the extending/retracting position (FIG. 5C) to the extended and use position (FIG. 5D).

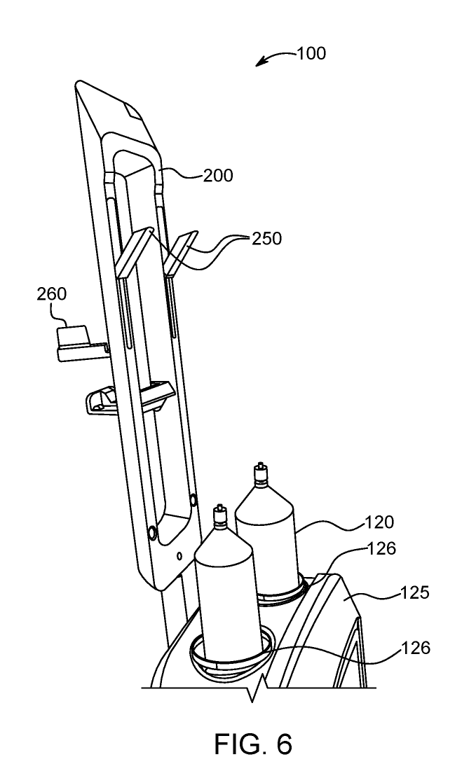

FIG. 6 shows an example of the retractable pole 200 of the injector 100 in the fully deployed, use position where the at least one hanging feature 250 is deployed vertically over the at least one syringe 120. In this position, one or more multi-dose bottles (not shown) may be hung from pivotable hooks 250 in an inverted position over the at least one syringe 120 and attached to the fluid port of the syringe 120 by a fluid path (not shown) to allow fluid communication and flow of medical fluid from the multi-dose fluid container to the at least one syringe 120. FIG. 6 further shows the rotatable shelf member 260 in a deployed position behind retractable pole 200. In this position, one or more fluid containers or other objects may be placed on the shelf 260 and located behind the retractable pole 200. In specific examples, the retractable pole 200 may be used as a handle for carrying or rolling the injection assembly when the handle is either in the fully retracted and locked position or in the fully extended and locked position.

FIG. 7 illustrates a system 1000 for controlling a fluid delivery in accordance with a non-limiting example of the present disclosure. In the non-limiting example shown, a fluid delivery device 136 comprising at least one processor may be formed as part of or connected to the housing 125 and configured or programmed to control an injection and/or fluid delivery in a syringe 120 engaged in a syringe port 126.

In the non-limiting example shown, the fluid delivery device 136 is in wired and/or wireless communication, as described herein, with a portable computer 150, a network 162, a patient records database 164, a plurality of syringe identification sensors 184, and a secondary controller 182. In further non-limiting examples, the portable computer 150 may be further configured to be in wired or wireless communication with a medical scanner, an injection protocol database, or other device or system and to allow a user to download, upload, display, and/or manipulate data from the medical scanner, injection protocol database, and/or other systems related to the medical injection procedure.

In non-limiting examples, the secondary controller may comprise a plurality of buttons, a touch screen comprising a GUI, or other input mechanism and may provide a user with at least a subset of the functionality compared to that which is available using the portable computer 150. In non-limiting examples, the secondary controller 182 may be located on or connected to the housing 125 and is configured to provide a user with limited control options, for example, when the portable computer 150 is unavailable. In non-limiting examples, the subset may include the ability to start and/or stop an injection procedure. In non-limiting examples, the secondary controller 182 may, for example, function as a back-up or emergency stop or abort, for situations in which the portable computer 150 is not immediately available or within reach. In further non-limiting examples, the secondary controller 182 may also provide the user with the ability to initiate an injection, change a flow rate of an injection, or perform other like procedures.

In the non-limiting example shown in FIG. 7, the housing 125 is further provided with a docking station 142 adapted to releasably engage the portable computer 150 and comprising a port 141 which may provide the portable computer 150 with a wired connection when the portable computer 150 is engaged in the docking station 142. Attachment/docking of the portable computer 150 with the docking station 142 may, in non-limiting examples, be via a mechanical attachment mechanism or a magnetic attachment mechanism. In the non-limiting example shown, the docking station 142 is positioned such that the portable computer 150 obscures at least a portion of the secondary controller 182 when engaged therein, however, optionally, the secondary controller 182 may remain visible and/or usable when the portable computer 150 is docked. The secondary controller may optionally be further configured or programmed to be inactive when the portable computer 150 is engaged within the docking station 142. This configuration allows for access to, for example, redundant control options provided on the secondary controller 182, in the absence of the portable computer 150, and helps to prevent confusion or unauthorized access to the system when the portable computer 150 is present in the docking station.

In the non-limiting example of FIG. 7, the fluid control device 136 may be provided with a syringe detection unit 184 associated with each syringe port 126 and in communication with the processor 136 of the fluid control device 136 as well as with the portable computer 150. Each syringe detection unit 184 may comprise a sensor such as a camera, a radio frequency receiver, an optical label reader, a magnetic sensor, an optical sensor, a mechanical sensor, or any combination thereof, which is configured to detect identifying information about a syringe 120 engaged within its corresponding syringe port and communicate this information to the processor 136 and/or portable computer 150.

Various syringes 120 intended for use with the present disclosure may be equipped with corresponding identity indicators to facilitate detection by the syringe detection unit. In non-limiting examples, a type of syringe may be equipped with a retention feature comprising a plurality of grooves or protrusions, which are arranged in a specific identifying pattern that is distinguishable by a mechanical sensor. In further non-limiting examples, the syringe may be equipped with a magnetic tag, a bar code, a label such as a barcode or QR code, or other indicator which provides information about the syringe 120. In further non-limiting examples, the syringe may have a distinguishing shape or appearance which a syringe detecting unit 184 comprising a camera in communication with a processor comprising appropriate image recognition software can use to identify the syringe type, dosage, the presence or absence of air bubbles, an indication that the syringe is loaded properly, and/or other identifying properties, such as to indicate whether the syringe has been previously used, to indicate whether the syringe has passed its useful shelf-life expiration date. Additional identifying properties may include a syringe lot number, a manufacture date, and/or a facility of manufacture.

The fluid control device 136, the portable computer 150, or another computer in communication with the syringe detection unit 184 may be configured or programmed to recall a pre-programmed injection protocol based on information obtained from the syringe detection unit 184, for example, from an internal memory, from a network 162, or from an injection protocol database. Additionally or alternatively, the fluid control device 136 and/or portable computer 150 may be configured or programmed to provide an alert and/or warning and/or to prevent an injection from occurring if the syringe is determined to be an unauthorized type incompatible, or previously used, or if not loaded properly into the injector. In certain non-limiting examples, the portable computer 150 and/or the fluid control device 136 may be further programmed or configured to require a manual override command from an authorized user (as determined using a user identifying information detector, described herein), and, upon receipt of the override command, allow a user to acknowledge the alert or warning and to initiate an injection despite the alert or warning. In such cases, the portable computer 150 and/or fluid control device 136 may be further configured or programmed to automatically record that an override command was received and various parameters relating to any injection that was performed thereafter, and to communicate the resulting record to a hospital network, a third party network, and/or another computer in communication with the system for historical and/or monitoring purposes.

In further non-limiting examples, the system may comprise at least one user identifying information detector in communication with and/or integral to at least one of the fluid control device 136 and the at least one portable computer 150. At least one of the fluid control device 136 and the at least one portable computer 150 may be further configured or programmed to selectively allow an identified user to view and/or manipulate injection parameters and/or cause the fluid control device 136 to initiate an injection based at least partially on data received from the at least one user identifying information detector.

By way of example, the user identifying information detector may include at least one of the following: a biometric scanner, an ID card reader, a microphone, a password protected machine readable-medium, a portable computer identity sensor configured or programmed to determine which of a plurality of portable computers is providing instructions to the fluid control device, or any combination thereof. In one non-limiting example, the user identifying information detector is integral to one of the portable computer and the secondary controller.

In other non-limiting examples, the fluid control device and/or the portable computer may be programmed or configured to identify users of different access levels and to allow access of various functions to only authorized users of certain levels. In a non-limiting example, a specific physician may be granted access only to information corresponding to his or her patients and denied access to information regarding a second physician's patients. In a further non-limiting example, a supervisor-level access may be required in order to initiate an override command if a syringe detection unit detects that a syringe is of an unusual or normally-unauthorized type or if there is an abnormality in the way the syringe 120 is loaded. In further non-limiting examples, lower-level users may be enabled to monitor and view injection data, and/or initiate an emergency stop, but may be prevented from initiating an injection and/or changing certain injection parameters. In other embodiments, there may be multiple portable computer 150 devices, for example, assigned to specific users of the fluid injector that may be in communication with the fluid control device 136 at any given time. In specific embodiments, the system may be configured to allow only one authorized user to operate the fluid control device 136 and perform an injection procedure at a given time and to lock out all other users from accessing or changing certain features of the injection procedures until a specific injection procedure is completed. In certain embodiments, the lock-out feature to prevent more than one portable computer 150 from controlling the fluid control device 136 at a given time may be overridden by an authorized user having a sufficient access level, such as a supervisor level.

In further non-limiting examples, the system may further comprise a proximity sensor in communication with the fluid control device 136 and the at least one portable computer 150. Based at least partially on information from the proximity sensor, the fluid control device may be further configured or programmed to prevent a user from accessing certain functionality if the portable computer is located at a distance further than a predetermined threshold (e.g., safe control distance) from the injector. Alternatively or additionally, at least one of the portable computer 150 and the fluid control device 136 may be configured to provide a user with a warning, such as a visual indicator on a display or an audible signal indicating that the portable computer 150 is outside of a safe control distance from the injector 100. In a non-limiting example, the fluid control device may be configured to communicate with the portable computer via a radio frequency protocol of a known range such as Bluetooth or NFC and be configured to only accept injection instructions if the signal strength is at a predetermined level which indicates that the portable computer is within a safe control distance. In a further non-limiting example, the location of the fluid control device and/or at least one portable computer may be determined using local network triangulation or GPS technology and the fluid control device and/or at least one portable computer may be further configured to limit access if the portable computer is determined to be outside of a safe control distance from the fluid control device 136. It will be appreciated that other configurations are possible in accordance with the present disclosure.

Alternatively or additionally, in a non-limiting example one of the fluid control device and the at least one portable computer may be configured or programmed to prevent a user from accessing certain functionally when the at least one portable computer is not engaged in the docking station. For example, a user may be enabled to view, monitor, and input various injection parameters while the portable computer is detached, but only able to initiate an injection when the portable computer is engaged in the docking station.

1. A system for delivering fluid to a patient, the system comprising:
an injector housing comprising at least one syringe port adapted to releasably engage at least one syringe; and
a fluid control device adapted to interface with and actuate at the least one syringe engaged in the at least one syringe port, the fluid control device comprising at least one processor programmed or configured to control a fluid delivery of at least one fluid to at least one patient and to communicate with at least one portable computer;
wherein the at least one portable computer is programmed or configured to receive or deliver at least one fluid delivery parameter from or to at least one of the fluid control device, a user interface, an imaging device, a patient records database, a second computer, a server, a hospital network, a wireless network, or any combination thereof and generate a display comprising information pertaining to the at least one fluid delivery parameter based at least partially on the received and/or delivered data; and
wherein the fluid control device is configured or programmed to execute at least one control option based at least partially on instructions received or delivered from the at least one portable computer.

2. The system according to claim 1, wherein the at least one portable computer comprises at least one of a laptop computer, a tablet computer, a smartphone, a personal data assistant device, a handheld computer, and any combination thereof.

3. The system according to claim 1, wherein the at least one portable computer is further configured to be in at least one of wireless and wired, communication with the fluid control device.

4. The system according to claim 1, wherein the housing is rotatably connected to a support portion and wherein at least one of the housing and the support portion further comprises a docking station adapted to releasably engage the at least one portable computer.

5. The system according to claim 4, wherein the docking station further comprises a port adapted to interface with the at least one portable computer when the at least one portable computer is engaged in the docking station and to provide the at least one portable computer with at least one wired connection to at least one of the fluid control device, a user interface, an imaging device, a patient records database, a second computer, a server, a power source, or any combination thereof.

6. The system according to claim 5, wherein:
the docking station is connected to the housing such that it is rotatable along with the housing relative to the support portion;
the display is generated on at least one of the portable computer and the housing; and
at least one of the housing, the support portion, and the at least one portable computer further comprises an orientation sensor configured or programmed to detect an orientation of the at least one portable computer at least while the at least one portable computer is engaged in the docking station, or, alternatively, while the at least one portable computer is in a disengaged, handheld state, and to communicate the orientation to the at least one portable computer,
wherein the at least one portable computer is programmed or configured to adjust an orientation of the generated display based at least partially on data received from the orientation sensor.

7. The system according to claim 6, wherein the orientation sensor comprises at least one of a gyroscopic sensor, a mechanical sensor, a magnetic sensor, an optical sensor, or any combination thereof, and wherein the at least one portable computer is programmed or configured to continuously or discontinuously adjust the orientation of the generated display to an upright orientation during a rotation of the housing.

8. The system according to claim 1, wherein the at least one portable computer comprises at least one of a circular touch screen, an oval touch screen, an elliptical touch screen, and a quadrilateral touch screen.

9. The system according to claim 1, wherein the at least one portable computer is adapted to releasably engage with a docking station attached to the housing via at least one of a mechanical connection and a magnetic connection.

10. The system according to claim 9, wherein the at least one portable computer further comprises a rechargeable battery configured to charge when the at least one portable computer is engaged with the docking station.

11. The system according to claim 1, wherein:
the at least one control option comprises at least one of initiating a delivery of the at least one fluid, stopping a delivery of the at least one fluid, changing a rate of a delivery of the at least one fluid, recalling and/or executing a pre-programmed fluid delivery protocol of the at least one fluid, delivering a specified volume of the at least one fluid, delivering the at least one fluid in a specified sequence and/or ratio, or any combination thereof.

12. The system according to claim 1, further comprising:
a secondary controller in communication with the fluid control device;
wherein the secondary controller is connected to or forms a part of the housing; and
wherein, the secondary controller is configured or programmed to carry out at least a subset of the at least one control option, including at least one of initiating a delivery of the least one fluid and stopping a delivery of the at least one fluid.

13. The system according to claim 12, wherein the secondary controller is arranged such that when the at least one portable computer is engaged within the docking station, the secondary controller is at least partially physically obscured or rendered inoperable.

14. The system according to claim 1, further comprising at least one syringe detection unit in communication with at least one of the fluid control device and the at least one portable computer, wherein the syringe detection unit is configured or programmed to detect at least one identifying property of the at least one syringe and to communicate the at least one identifying property to at least one of the fluid control device and the at least one portable computer.

15. The system according to claim 14, wherein at least one of the fluid control device and the at least one portable computer is further configured or programmed to at least one of: prevent a fluid delivery from occurring, provide an alert when a non-compatible, previously used, or non-approved syringe is attached to the at least one syringe port, require a user override when a non-compatible, previously used, or non-approved syringe is attached to the at least one syringe port, and recall at least a portion of a pre-programmed fluid delivery protocol, based at least partially on data received from the syringe detection unit, or any combination thereof.

16. The system according to claim 14, wherein the syringe detection unit comprises at least one of a camera, a radio frequency receiver, an optical label reader, a magnetic sensor, an optical sensor, a mechanical sensor, or any combination thereof, wherein at least one of the fluid control device and the at least one portable computer is further configured or programmed to prevent a fluid delivery from occurring unless the at least one identifying property comprises at least one predetermined authorized property or an override command is received from an authorized user.

17. The system according to claim 14, wherein the at least one identifying property comprises at least one of an indication of the type of syringe, and indication of the brand of the syringe, an indication that the syringe is loaded to a correct volume of fluid, an indication that the syringe has been previously used, an indication whether the syringe has passed its useful shelf-life, an expiration date, a lot number, a manufacture date, a facility of manufacture, or any combination thereof.

18. The system according to claim 1, further comprising at least one user identifying information detector in communication with or integral to at least one of the fluid control device and the at least one portable computer, wherein at least one of the fluid control device and the at least one portable computer is further configured or programmed to selectively allow an approved, identified user to cause a fluid delivery to occur based at least partially on data received from the at least one user identifying information detector.

19. (canceled)

20. (canceled)

21. The system according to claim 1, wherein the at least one fluid delivery parameter comprises one or more of a fluid delivery instruction, a fluid delivery flow rate, a fluid delivery duration, a total fluid delivery volume, a remaining volume, a ratio of a first injected fluid to a second injected fluid, a fluid delivery fluid concentration, a fluid delivery fluid identity, a sequence and/or ratio for delivering at least two fluids, a pre-programmed fluid delivery protocol, or any combination thereof.

22. An injector assembly, comprising
a housing comprising at least one syringe port adapted to releasably engage at least one syringe;
a fluid control device adapted to interface with and actuate at least one syringe engaged in the at least one syringe port, the fluid control device comprising at least one processor programmed or configured to control a fluid delivery and to communicate with at least one portable computer; and
a support portion comprising a retractable pole configured to reversibly move from a retracted position to a deployed position to extend above the injector assembly, the retractable pole comprising at least one hanging feature for hanging at least one multi-dose fluid container.

23. (canceled)

24. (canceled)
